(19) United States
(12) Patent Application Publication
(10) Pub. No.: US 2017/0021002 A1
(43) Pub. Date: Jan. 26, 2017

(54) AUGMENTATION OF CANCER AND CANCER ENDOTHELIAL VACCINE IMMUNOGENICITY BY HISTONE DEACETYLASE INHIBITORS

(71) Applicant: Batu Biologics, Inc., San Diego, CA (US)

(72) Inventors: Samuel C. Wagner, San Diego, CA (US); Thomas E. Ichim, San Diego, CA (US); Vladimir Bogin, San Diego, CA (US); Boris Minev, San Diego, CA (US)

(73) Assignee: Batu Biologics, Inc., San Diego, CA (US)

(21) Appl. No.: 15/158,476

(22) Filed: May 18, 2016

Related U.S. Application Data
(60) Provisional application No. 62/162,952, filed on May 18, 2015.

(51) Int. Cl. A61K 39/00; A61K 31/19

(52) U.S. Cl. CPC A61K 39/0011; A61K 31/19; A61K 2039/515

(57) ABSTRACT

Disclosed are protocols, procedures and therapeutic compositions useful for augmentation of immunity to cancer and cancer associated endothelial cells by treatment with histone deacetylase (HDAC) inhibitors capable of augmenting stimulatory and costimulatory molecules on said cancer vaccines. Additionally, the invention teaches specific concentrations of HDAC inhibitors useful for stimulation of in vivo immunity to tumor and tumor endothelial cell targeting vaccines.

### CROSS-REFERENCE TO RELATED APPLICATION

This application claims the benefit of U.S. Provisional Application No. 62/162,952 filed on May 18, 2015, the contents of which are incorporated herein by reference in its entirety.

### BACKGROUND

In the USA, lung cancer deaths per annum are higher than breast cancer, colon cancer, and melanoma combined. Approximately 80-85% of the newly diagnosed cases of lung cancer are non-small cell lung cancer (NSCLC) (adenocarcinoma, squamous carcinoma, and large cell carcinoma) and 15-20% small cell lung carcinoma. In the majority of cases, patients present with unresectable and/or non-curable disease. Locally advanced, good performance status NSCLC patients may be offered concurrent chemotherapy, radical radiotherapy, and/or surgery, with a resultant 8-month progression-free survival rate and <15% 5-year survival. Patients diagnosed with metastatic disease newer cytotoxic chemotherapies such as pemetrexed [17-month median overall survival (OS)] and treatment with molecularly targeted therapeutics for adenocarcinomas, such as next generation small molecules targeting the EGFR (24 months median OS) and ALK inhibitors (20 months median OS), the survival rate for advanced disease has improved only marginally. In the last decade, there has been a better understanding on how cancer interacts with the immune cells and the ways that the cancer have developed to evade the immune system, resulting in a new era of cancer immunotherapy protocols, which may aid in overcoming the limitations of conventional therapeutic strategies.

Unfortunately, targeting of tumor cells themselves by immunotherapy possesses the following drawbacks: a) inability of immune cells to physically enter the tumor due to high tumor interstitial pressures; b) intratumor acidosis which limits activity of immune cells; and c) genetic instability of the tumor, which allows for antigenic shift and antigen loss after immune pressure. Targeting of proliferating endothelial cells in cancer therapy is a clinically validated approach as evidenced by the success of agents such as the vascular endothelial growth factor (VEGF-targeting antibody Bevacizumab. Unfortunately, long term success of such passive anti-angiogenic immunotherapy is limited by lack of antibody cytotoxicity to tumor endothelium, by need for repeat administrations, which often possesses adverse effects, and by development of resistance.

Active immunization against tumor endothelium by vaccinating against proliferating endothelium or markers found on tumor endothelium has provided promising preclinical data. Specifically, in animal models it has been reported that immunization to antigens specifically found on tumor vasculature can lead to tumor regression. Studies have been reported using the following antigens: survivin, endosialin, and xenogeneic FGF2R, VEGF, VEGF-R2, MMP-2, and endoglin. Human trials have been conducted utilizing human umbilical vein endothelial (HUVEC) cells as tumor antigens, with responses being reported in patients. In one report describing a 17-patient trial, Tanaka et al demonstrated that HUVEC vaccine therapy significantly prolonged tumor doubling time and inhibited tumor growth in patients with recurrent glioblastoma, inducing both cellular and humoral responses against the tumor vasculature without any adverse events or noticeable toxicities.

### SUMMARY

To our knowledge, there is only one commercial entity developing an anti-angiogenic vaccine. This vaccine, ValloVax™, is a placenta endothelium-derived therapeutic vaccine, which has reported therapeutic efficacy in animal models of lung cancer, breast cancer, and melanoma. ValloVax™ was granted an IND # by the FDA and is currently being developed for the treatment of non-small cell lung cancer. As previously reported, one of the advantages of ValloVax™ in comparison to other tumor endothelium targeting vaccines is the immunogenicity of the vaccine, which is endowed by interferon gamma pretreatment. In this study we sought to enhance immunogenicity by assessing different agents that are clinically utilized. We found valproic acid treatment was associated with killing of ValloVax™ in vitro by an NK cell dependent mechanism, and while in vitro treatment of ValloVax™ did not augment in vivo efficacy, in vivo treatment of animals receiving ValloVax™ augmented efficacy against lung cancer.

Still other advantages, aspects and features of the subject disclosure will become readily apparent to those skilled in the art from the following description wherein there is shown and described a preferred embodiment of the present disclosure, simply by way of illustration of one of the best modes best suited to carry out the subject disclosure As it will be realized, the present disclosure is capable of other different embodiments and its several details are capable of modifications in various obvious aspects all without departing from the scope herein. Accordingly, the drawings and descriptions will be regarded as illustrative in nature and not as restrictive.

### BRIEF DESCRIPTION OF THE FIGURES

The accompanying drawings incorporated herein and forming a part of the specification illustrate the example embodiments.

FIGS. 1a-1d show the effects of valproic acid and interferon gamma treatment on ValloVax™.

FIGS. 2-4 illustrate the viability of valproic acid treated ValloVax™.

FIG. 5 illustrates the effects of in vivo treatment of valproic acid on ValloVax™.

### DETAILED DESCRIPTION OF THE EMBODIMENTS

This description provides examples not intended to limit the scope of the appended claims. The figures generally indicate the features of the examples, where it is understood and appreciated that like reference numerals are used to refer to like elements. Reference in the specification to “one embodiment” or “an embodiment” or “an example embodiment” means that a particular feature, structure, or characteristic described is included in at least one embodiment described herein and does not imply that the feature, structure, or characteristic is present in all embodiments described herein.

It has previously been reported that ValloVax™ a placenta-derived endothelial cell vaccine, induces immunity to lung cancer, breast cancer, and melanoma by inhibiting tumor derived angiogenesis. In an attempt to augment therapeutic efficacy of ValloVax™ we pretreated the placental derived endothelial cells with valproic acid, a clinically-used histone deacetylase inhibitor (HDAC). In mixed lymphocyte reactions we observed that valproic acid pretreated ValloVax™ would elicit spontaneous cytotoxicity by NK cells in responding lymphocytes. When valproic acid treated ValloVax™ was used to immunize Lewis Lung Carcinoma (LLC) bearing mice, no enhancement of therapeutic efficacy was observed compared to standard ValloVax™. In vivo treatment of animals with valpoic acid resulted in enhanced antitumor efficacy. NK cells isolated from in vivo valproic acid treated mice possessed enhanced cytotoxicity to ValloVax™ cells ex vivo, as well as to LLC cells. These data suggest modulation of NK cells may be a possible means to enhance efficacy of tumor endothelium targeting immunotherapy.

A variety of TAAs have been identified in lung cancer consisting of overexpressed normal proteins and mutated proteins that are normally found in pulmonary tissue, however, only a minority of the TAAs that have been discovered so far are immunogenic, which limits the potential use for immunotherapy. In addition, while the overwhelming majority of TAAs are expressed in tumor cells, they are typically also expressed in a variety of normal cells, e.g. the lung cancer TAAs; epidermal growth factor receptors (HER2), carcinoembryonic antigen (CEA), mucin (MUCI), the tumor suppressor protein p53, and telomerase reverse transcriptase (TERT). In one embodiment of the invention these TAA are utilized as immunogens, to be administered concurrently with ValloVax™ and VPA.

It is important for the practice of the invention since TAA are recognized by the immune system as self-molecules, and the immune system has protective mechanisms for preventing recognition of self-tissue antigens and autoimmune responses. Additionally, tumors employ other mechanisms for escaping immune surveillance, such as: (i) low level expression of MHC class I molecules; (ii) lack of expression of B7 (CD80/CD86) co-stimulatory molecules; (iii) production of cytokines that stimulate the accumulation of immune-suppressor cells; and (iv) ineffective processing and presentation of self-antigens by “professional” antigen-presenting cells (APC).

### EXAMPLES

### Materials and Methods

### Animals and Cells

Female C57BL/6 aged 8-12 weeks were purchased from The Jackson Laboratory. Animals were housed under conventional conditions at the Animal Care Facility, Institute for Cellular Immunology, and were cared for in accordance with the guidelines established by the Canadian Council on Animal Care. Lewis Lung Carcinoma (LLC), a murine lung carcinoma originating from C57/BL6 mice was obtained from American Type Culture Collection (ATCC). The cells were maintained in RPMI1640 supplemented with 10% fetal bovine serum, 2 mM glutamine (Gibco-BRL, Life Technologies, Inc.) and were passaged by trypsinization once cells reached 75% confluence. The cell line was cultured at 37°0 C. in a 5% CO2 incubator under fully humidified conditions.

### Preparation of Vaccine

The protocol described by Ichim et al was followed. Full term human placentas were collected from delivery room under informed consent. Fetal membranes were manually peeled back and the villous tissue is isolated from the placental structure. Villous tissue was subsequently washed with cold saline to remove blood and scissors used to mechanically digest the tissue. Lots of 25 grams of minced tissue were incubated with approximately 50 ml of HBSS with 25 mM of HEPES and 0.28% collagenase, 0.25% dispase, and 0.01% DNAse at 37 Celsius. The mixture of minced placental villus tissue and digesting solution was incubated under stirring conditions for three incubation periods of 20 minutes each. Ten minutes after the first incubation period and immediately after the second and third incubation periods, the DNAse was added to make up a total concentration of DNase, by volume, of 0.01%. In the first and second incubations, the incubation flask is set at an angle, and the tissue fragments allowed to settle for approximately 1 minute, with 35 ml of the supernatant cell suspension being collected and replaced by 38 ml (after the first digestion) or 28 ml (after the second digestion) of fresh digestion solution. After the third digestion the whole supernatant was collected. The supernatant collected from all three incubations was then pooled and is poured through approximately four layers of sterile gauze and through one layer of 70 micrometer polyester mesh. The filtered solution was then centrifuged for 1000 g for 10 minutes through diluted new born calf serum, said new born calf serum diluted at a ratio of 1 volume saline to 7 volumes of new born calf serum. The pooled pellet was then resuspended in 35 ml of warm DMEM with 25 mM HEPES containing 5 mg DNase I. The suspension was subsequently mixed with 10 ml of 90% Percoll to give a final density of 1.027 g/ml and centrifuged at 550 g for 10 minutes with the centrifuge brake off. The pellet was then washed in HBSS and cells incubated for 48 hours in complete DMEM media. After 3-4 passages cells were incubating in media containing 100 IU of IFN-gamma per mi. Subsequent to incubation cells were either used: a) unmanipulated; b) used as a lysate, with 10 freeze thaw cycles in liquid nitrogen, subsequent to which lysate was filtered through a 0.2 micron filter; c) mitotically inactivated by irradiation at 10 Gy; or d) inactivated by fixation in 0.5% formalin and subsequently washed.

### In Vitro Treatment With VPA

Valproic acid (Sigma-Aldrich, St. Louis, Mo., USA). Bovine serum albumin (BSA) and trypsin were purchased from Amresco, Solon, Ohio, USA. Fetal bovine serum (FBS), donor equine serum (DES), Alpha modified eagle medium (alpha-MEM), and Dulbecco's modified eagle medium F12 (DMEM/F12) were obtained from Hyclone, Logan, Utah, USA.

Cells were incubated with or without 1 mM VPA for 48 hours.

NK-92 cells were added to the target cells as effector cells, and the cells were co-cultured for 4 h 37° C. To block NKG2D on NK-92 cells, 10 μg/ml anti-NKG2D mAb or mouse IgG1 isotype control antibody were added to the NK cells 30 min before co-culture.

Depletion of T cells, B cells and NK cells was performed with Magnetic Activated Cell (MACS) isolation kits from Milteny Biotec following the manufacturer's instructions.

Viability was assessed by CellTiter Viability kit from Promega following the manufacturer's instructions.

### Mixed Lymphocyte Reaction and ELISA

Peripheral blood mononuclear cells (PBMC) were isolated from buffy coats of healthy blood donors (Sanquin, Rotterdam, the Netherlands) by density gradient centrifugation using Ficoll-Paque PLUS (density 1.077 g/ml; GE Healthcare, Uppsala, Sweden). Cells were frozen at −150° C. until further use in RPMI-1640 medium with GlutaMAX™-I (Life Technologies) supplemented with 1% P/S, 10% human serum (Sanquin) and 10% dimethylsulphoxide (DMSO; Merck, Hohenbrunn, Germany).

Mixed lymphocyte reactions (MLR) were set up with 5×10⁵ responder PBMC and 5×10³ (1:100), 5×10⁴ (1:10), 5×10³ (1:1) γ-irradiated (10 Gy) ValloVax™ cells in round-bottomed 96-well plates (Nunc, Roskilde, Denmark). MLR were cultured in MEM-α supplemented with 2 mM L-glutamine, 1% P/S and 10% heat-inactivated human serum for 4 days in a humidified atmosphere with 5% CO₂ at 37° C.

Cell proliferation was assessed by thymidine incorporation, [³H]-thymidine (0.25 μCi/well; PerkinElmer, Groningen, the Netherlands) was added on day 4, incubated for 8 h and its incorporation was measured using the Wallac 1450 MicroBeta Trilux (PerkinElmer).

For cytokine analysis supernatant was collected on day two of culture and analyzed by ELISA (R & D Systems) as per manufacturer's instructions.

### Immunization Schedules and Tumor Assessment

For induction of tumor growth, 5×10⁵ LLC cells, American Type Culture Collection (Manassas, Va.) cells were injected subcutaneously into the hind limb flank. Four weekly vaccinations of 5×10⁵ test cells were administered subcutaneously on the contralateral side to which tumors were administered. Tumors were allowed to grow for 2 weeks, subsequently to which one injection of ValloVax™ or VPA-pretreated ValloVax™ was given. Valproic acid was administered every third day at a concentration of 100 mg/kg intraperitoneally. Tumor growth was assessed every 3 days by two measurements of perpendicular diameters by a caliper, and animals were sacrificed when tumors reached a size of 1 cm in any direction. Tumor volume was calculated by the following formula: (the shortest diameter²×the longest diameter)/2.

### Results

### VPA Stimulates Allogenicity of Placental Derived Endothelial Cells Cultured in Interferon Gamma (ValloVax™)

It was previously reported that ValloVax™, a placental endothelial derived cellular vaccine stimulates immunity to proliferating endothelium, resulting in tumor regression. Although the previous publication reported induction of superior immunity utilizing interferon gamma pretreatment of endothelial cells, as compared to untreated cells, the formal demonstration that the interferon gamma pretreatment actually increases allogenicity was not reported. Accordingly, we performed mixed lymphocyte reaction using escalating concentrations of PBMC mixed with one concentration irradiated stimulatory cells, said stimulatory cells comprising of a) placental endothelial cells; b) placental endothelial cells cultured with interferon gamma; c) placental endothelial cells cultured with VPA; and d) placental endothelial cells cultured with interferon gamma and VPA.

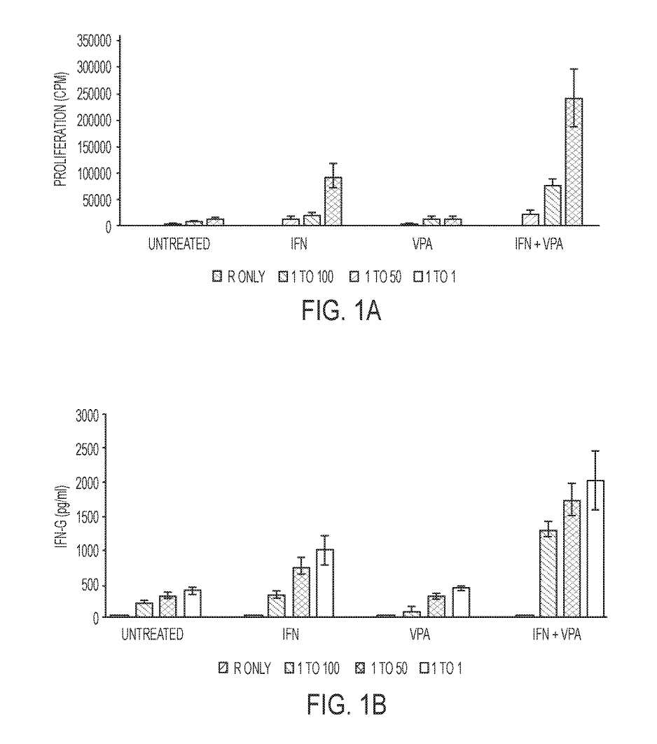

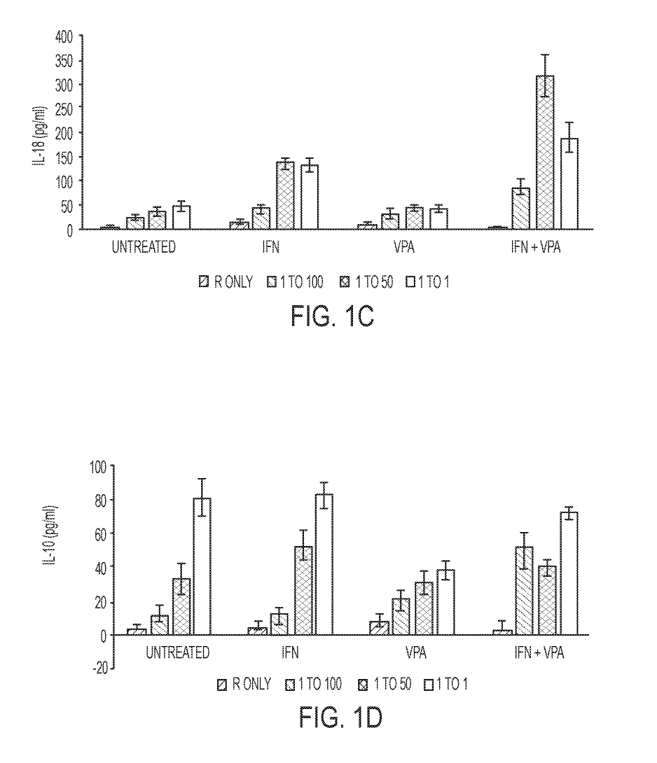

Proliferation of allogeneic responding lymphocytes was substantially enhanced by pretreatment with interferon gamma, but not with VPA. Interestingly the combination of VPA and interferon gamma led to a profound increase in allostimulatory activity, substantially higher than the interferon gamma pretreatment alone (FIG. 1a).

### VPA Plus IFN-Gamma Endow Placental Endothelial Cells with Ability to Stimulate NK Promoting Cytokine Responses

One of the potential mechanisms by which ValloVax™ exerts its antitumor effects is through stimulation of cytotoxic T cell responses towards tumor endothelium. Accordingly, we sought to detect whether the addition of VPA would augment production of relevant cytokines in the mixed lymphocyte reaction. Collection of supernatants from MLR at 48 hours revealed that treatment of ValloVax™ with VPA substantially increased production of the NK stimulating cytokines IFN-gamma (FIG. 1b) and IL-18 (FIG. 1c). Once potential concern was that VPA may be stimulating T regulatory cell production, which was previously described in the literature. When the T regulatory cell stimulatory cytokine IL-10 was assessed in the MLR, no significant upregulation was observed (FIG. 1d).

### VPA Treatment of ValloVax™ Induces NK-Mediated Killing of Stimulator ValloVax™ Cells in MLR

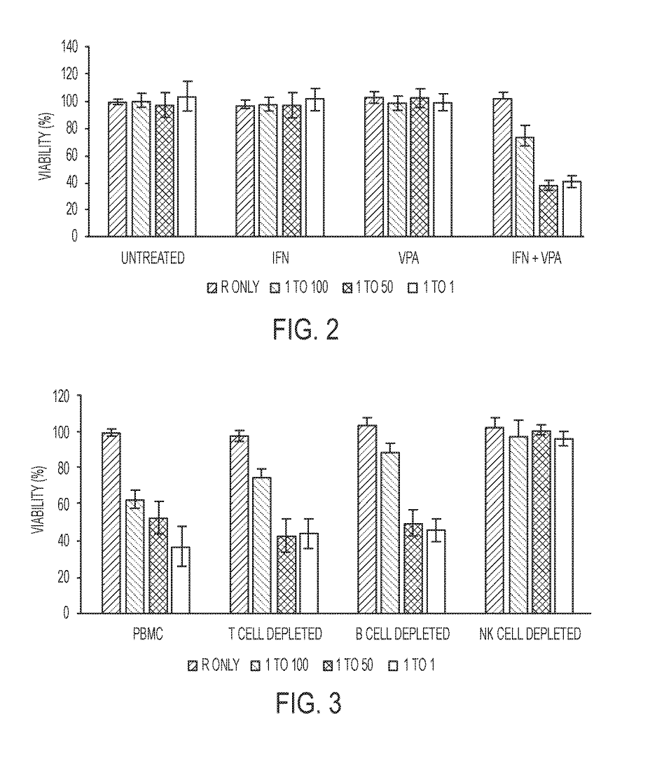

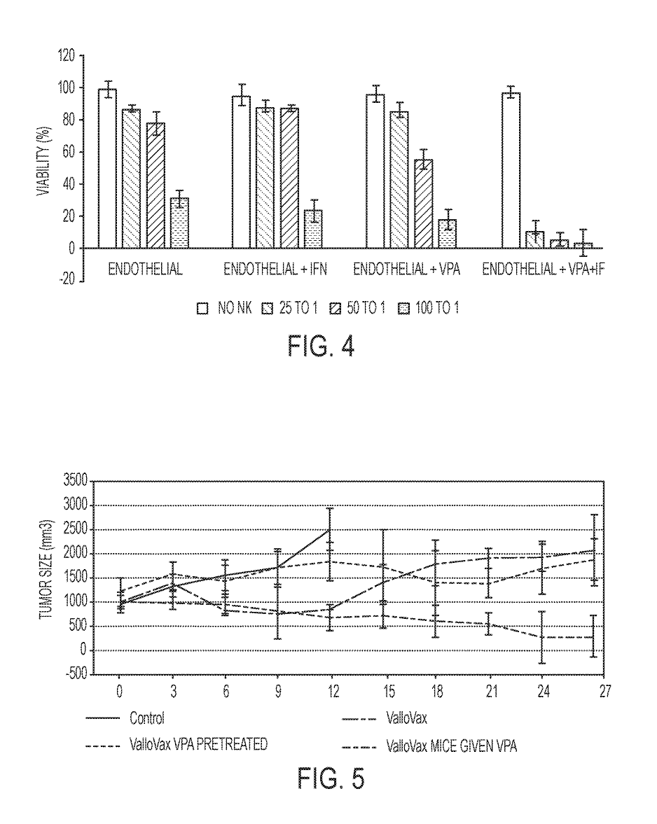

Based on visual examination, it appeared that the adherent cells in the MLR experiments described above were losing viability as the culture was progressing. Accordingly, viability of the ValloVax™ cells was assessed. As seen in FIG. 2, a dose-dependent loss of viability was observed in the ValloVax™ cells treated with VPA. Depletion studies demonstrated that the NK component of the allogeneic responding cells in the MLR were responsible for the killing of the ValloVax™ cells (FIG. 3). In order to validate using an independent model whether indeed VPA endows ValloVax™ cells with ability to be killed by NK cells, VPA treated ValloVax™ cells were exposed to the commercially available NK cell line NK-92. Indeed toxicity was observed when VPA treated ValloVax™ cells were cultured with NK-92 cells (FIG. 4).

### In Vivo Administration of VPA and ValloVax, but Not Administration of VPA Treated ValloVax™ Cells Significantly Enhances Survival in Established Lung Cancer Model

Given the demonstration of enhanced immunogenicity of ValloVax™ treated with VPA, we sought to determine whether administration of these cells in vivo would result in decreases in tumor growth in an established tumor model. As seen in FIG. 5, while pretreatment of ValloVax™ with VPA did not significantly augment tumor killing activity, synergistic antitumor activity was observed when VPA was systemically administered.

Having thus described certain embodiments of systems and methods for practicing aspects of the present disclosure, it is to be appreciated that various alterations, modifications, and improvements will readily occur to those skilled in the art. Such alterations, modifications, and improvements are intended to be part of this disclosure, and are intended to be within the spirit and scope of this disclosure.

1. A composition useful for induction of immune response to tumor associated endothelium produced by:
a) obtaining a population of endothelial cells;
b) inducing said endothelial cells to proliferate; and
c) treating said endothelial cells with a histone deacetylase inhibitor for a sufficient time and concentration to induce sensitivity of said proliferating endothelial cells to natural killer cell mediated killing.

2. The composition of claim 1, wherein said histone deacetylase inhibitor is valproic acid.

3. The composition of claim 2, wherein said valproic acid is used to culture cells for a period of approximately 48 hours at a concentration of approximately 1 milli Molar valproic acid.

4. The composition of claim 1, wherein said endothelial cells are derived from the placenta.

5. The composition of claim 1, wherein said endothelial cells are from the umbilical cord.

6. The composition of claim 1, wherein said endothelial cells are cultured in interferon gamma at a time and concentration sufficient to enhance immunogenicity of said endothelial cells.

7. The composition of claim 1, wherein said culture of endothelial cells is performed at a concentration of interferon gamma of 100 IU for a period of approximately 48 hours.

8. A method of treating cancer comprising the steps of:
a) administering an agent capable of stimulating an anti-tumor endothelial immune response; and
b) administering a histone deacetylase at a concentration and frequency sufficient to enhance said anti-endothelial cell response.

9. The method of claim 8, wherein said agent capable of stimulating said anti-endothelial response is ValloVax™.

10. The method of claim 9, wherein said ValloVax™ is administered at a concentration of approximately 25 million cells per injection, at a frequency of approximately every once every week for the first month, followed by monthly administration, said administration via subcutaneous route.

11. The method of claim 8, wherein said histone deacetylase inhibitor is selected from a group comprising of:
a) trichostatin A;
b) sodium butyrate; and
c) valproic acid.

12. The method of claim 11, wherein said histone deacetylase inhibitor is valproic acid.

13. The method of claim 12, wherein said valproic acid is administered at a concentration of approximately 100 mg/kg of body weight.

14. A method of stimulating NK cell activity in a patient comprising the steps of:
a) administering an agent capable of stimulating an anti-tumor endothelial immune response; and
b) administering a histone deacetylase at a concentration and frequency sufficient to enhance said anti-endothelial cell response.

15. The method of claim 14, wherein said agent capable of stimulating said anti-endothelial response is ValloVax™.

16. The method of claim 15, wherein said ValloVax™ is administered at a concentration of approximately 25 million cells per injection, at a frequency of approximately every once every week for the first month, followed by monthly administration, said administration via subcutaneous route.

17. The method of claim 14, wherein said histone deacetylase inhibitor is selected from a group comprising of:
a) trichostatin A;
b) sodium butyrate; and
c) valproic acid.

18. The method of claim 17, wherein said histone deacetylase inhibitor is valproic acid.

19. The method of claim 18, wherein said valproic acid is administered at a concentration of approximately 100 mg/kg of body weight.
